(19) United States
(12) Patent Application Publication
(10) Pub. No.: US 2019/0085382 A1
(43) Pub. Date: Mar. 21, 2019

(54) FTCD GENOTYPE AND GENE EXPRESSION USED TO PREDICT COGNITIVE ABILITY

(71) Applicant: Government of the United States as Represented by the Secretary of the Air Force, Wright-Patterson AFB, OH (US)

(72) Inventor: Kevin D. Schmidt, Dayton, OH (US)

(73) Assignee: Government of the United States as Represented by the Secretary of the Air Force, Wright-Patterson AFB, OH (US)

(21) Appl. No.: 16/136,669

(22) Filed: Sep. 20, 2018

Related U.S. Application Data
(60) Provisional application No. 62/561,468, filed on Sep. 21, 2017.

(51) Int. Cl. C12Q 1/6827; G16H 50/20; A61B 5/00; G16H 50/30; G16H 50/70; G16H 50/50

(52) U.S. Cl. CPC C12Q 1/6827; G16H 50/20; G16H 50/50; G16H 50/30; G16H 50/70; A61B 5/4088

(57) ABSTRACT

A method of predicting cognitive performance for a person, by acquiring a genetic sample from the person, determining an SNP of the rs914246 of the FTCD gene of the person, comparing the determined SNP against a cognitive model, and based on the comparison, returning a predicted cognitive performance.

### CROSS-REFERENCE TO RELATED APPLICATION

This application claims the benefit of and priority to prior filed U.S. Provisional Patent Application Ser. No. 62/561,468 (pending) filed Sep. 21, 2017, the entirety of the disclosure of which is incorporated herein by reference.

### RIGHTS OF THE GOVERNMENT

The invention described herein may be manufactured, used, and licensed by or for the Government of the United States for all governmental purposes without the payment of any royalty.

### FIELD OF THE INVENTION

This invention relates to the field of performance prediction assessments and, more particularly, this invention relates to cognitive performance prediction assessments.

### BACKGROUND OF THE INVENTION

Athletes, servicemen, law enforcement agents, and other professionals require a high level of cognitive ability to perform their duties in an efficient manner. This means that each individual's working memory must function at optimal levels to also function at optimal physical levels. Working memory is characterized as the part of an individual's short-term memory that is concerned with immediate, conscious perceptual and linguistic processing. Thus, an individual who is capable of processing information and retaining it in real-time more efficiently than others will be more suitable for professions that require rapid and immediate cognition, conscious perception, and memory.

Performance prediction assessments are used by athletic scouts, military recruiters, medical practitioners, and other agents to find suitable individuals to undertake jobs requiring such enhanced aptitude for job-related tasks. Conventional methods of performance prediction are largely based on physical measurement data and inferences based thereon. It is thus presumed that individuals having superior measurements will outperform individuals with inferior measurements.

According to some theories, working memory performance depends on sustained activity of neurons in the pre-frontal cortex, is modulated by extracellular dopamine, is related to dopamine D1 receptor binding, and is modulated by normal variation in genes that influence extracellular dopamine levels. Working memory tends to be strongly related to the individual's intelligence quotient, which is, in turn, linked to real-world abilities, morbidity, and even mortality.

At present, allelic association is the prominent theory in determining genetic influence on phenotypic expression, including that for working memory and cognitive ability. By understanding how genetic influences affect working memory, measurement and manipulation of cognitive ability may be possible.

Accordingly, there remains a need for genetic based performance models for predicting aptitude toward cognitive performance.

### SUMMARY OF THE INVENTION

The present invention overcomes the foregoing problems and shortcomings of conventional methods of assessing cognitive ability by physical and anatomical measurements. While the invention will be described in connection with certain embodiments, it will be understood that the invention is not limited to these embodiments. To the contrary, this invention includes all alternative, modifications, and equivalents as may be included within the spirit and scope of the present invention.

The above and other needs are met by a method of predicting cognitive performance for a person, by acquiring a genetic sample from the person, determining an SNP of the rs914246 of the FTCD gene of the person, comparing the determined SNP against a cognitive model, and based on the comparison, returning a predicted cognitive performance.

In various embodiment according to this aspect of the invention, the determination is of a C allele SNP. In some embodiments, the determination is of an A allele SNP. In some embodiments, a diet of at least one of increased folate and increased glutamate for the person is recommended. In some embodiments, correlation to physical measurements of the person are made. In some embodiments, expression of a C allele SNP of the rs914246 of the person is modulated. In some embodiments, cognitive training is recommended for the person. In some embodiments, DNA modification is recommended for the person.

Additional objects, advantages, and novel features of the invention will be set forth in part in the descript that follows, and in part will become apparent to those skilled in the art upon examination of the following or may be learned by practice of the invention. The objects and advantages of the invention may be realized and attained by means of the instrumentatalities and combination particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart illustrating a method according to an embodiment of the present invention.

FIG. 2 is a schematic illustration of an individual during a task illustrating a working memory load.

FIGS. 3A-3D is a schematic of a task illustrating a working memory load of three, according to an embodiment of the present invention.

FIG. 4 is a graph illustrating accuracy for the young female cohort at each target-test distance for a load of three as a function of rs914246 genotype, according to an embodiment of the present invention.

FIG. 5 is a graph of accuracy for the young male cohort at each target-test distance for a load of three as a function of rs914246 genotype, according to an embodiment of the present invention.

FIG. 6 is a chart of the mean Firefly luciferase activity for each of the four constructs of rs914245 and rs914246, according to an embodiment of the present invention. An asterisk, *, indicates a significant difference in Firefly luciferase activity. The GC haplotype had significantly higher luciferase activity than both the GA (p=0.02) and the TA haplotype (p=0.03), but was not significantly different from the TC haplotype (p=0.12)

FIG. 7 is a graph of the mean accuracy in the younger cohort as a function of haplotype and distance for a load of three, according to an embodiment of the present invention.

FIGS. 8A and 8B are ANOVA tables of Single SNP Effects on working memory accuracy, according to an embodiment of the present invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

Based on scientific study, the C allele of the rs914246 single nucleotide polymorphism (SNP) in the formimidoyltransferase cyclodeaminase gene (FTCD) has been associated with improved working memory performance in younger people (but not in older people). FTCD is a bifunctional enzyme that catalyzes the synthesis of the amino acid glutamate and certain forms of the vitamin folate, specifically formiminotetrahydrofolate from formiminoglutamate and tetrahydrofolate. Glutamate is recognized by some as the primary excitatory neurotransmitter in the brain and is critical for neural plasticity, spatial learning, and overall cognitive control. Accordingly, individuals expressing the FTCD genotype have a higher proclivity and aptitude for tasks involving substantial levels of cognition and intellect.

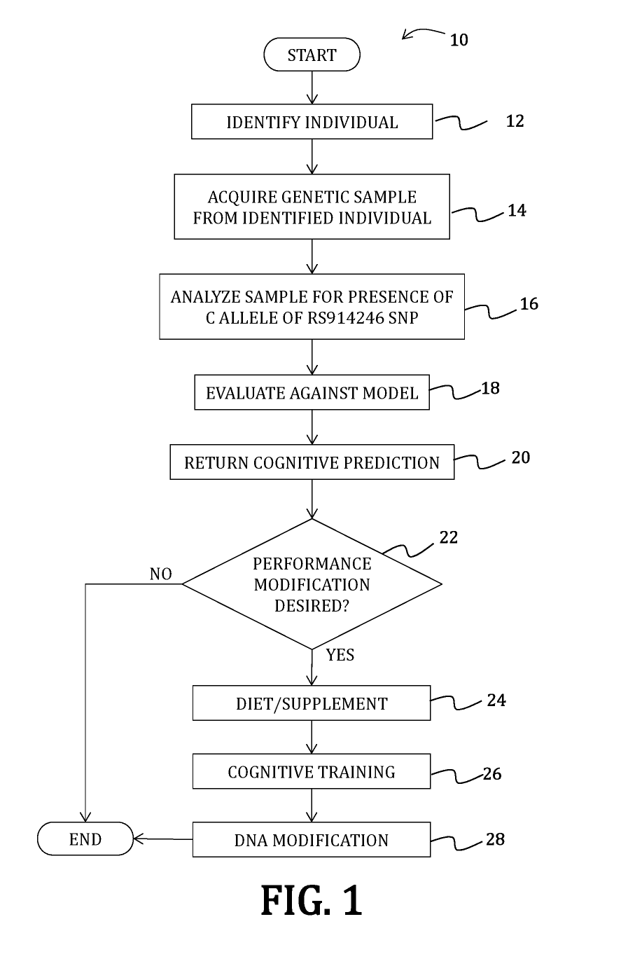

In use, and with reference now to FIG. 1, there is depicted a method 10 according to an embodiment of the present invention, which includes identifying an individual for performance prediction assessment (Block 12). The individual, and reason for assessment, may include, but are not limited to, athletic ability, military tacticians, military operatives, academic placement, predisposition to early cognitive disorders, and so forth.

With the individual identified, a genetic sample is acquired (Block 14). Genetic samples may include, but are not limited to, buccal sample, blood samples, hair samples, and so forth. DNA from such samples may be isolated, amplified, and sequenced according to methods known by those of ordinary skill in the art. One such method may include polymerase chain reaction.

In one embodiment, all DNA from the sample (the sample genome) is sequenced and analyzed for the presence of the common C allele of rs914246 (Block 16). According to other embodiments of the present invention, full sequencing of the individual's genome is not required—instead, a reporter gene assay may be used to investigate transcriptional regulation of a gene promotor. One specific embodiment of the present invention includes a haplotype based on the presence of the common C allele of rs914246, which is associated with increased reporter gene activity in transfected HEK293 cells. A reporter gene assay reveals the C allele of the rs914246 SNP to be a main factor regulating FTCD gene expression. A number of transcription factor binding sites are predicted for sequences flanking and including the polymorphic site defined by SNP rs914246.

While not wishing to be bound by theory, it is believed that two genes have roles in controlling levels of extracellular dopamine and affecting working memory performance in healthy older people, but not in younger people: catechol-O-methyltransferase (COMT VAL158MET) and dopamine betahydroxylase (DBH; C-1021T). High activity of the FTCD rs914246 C allele appears to encode an enzyme that is more efficient at converting formiminoglutamate and tetrahydrofolate into formiminotetrahydrofolate and glutamate, thereby presumably increasing levels of both substances. Because cognitive effects of the C allele are stronger in younger people, there may be some age-related decline in enzyme efficiency associated with age. Such age-related declines in gene expression have been reported in a range of tissues, including the brain.

In that regard, and according to one embodiment, the presence and activity of the C allele is evaluated against a model (Block 18), after which a cognitive level prediction may be returned (Block 20). For example, activity FTCD may be modeled as linearly related to working memory performance, then a level of activity for the individual may be determined. However, non-linear models may also be used and will depend, in part, on age-related decline of working memory performance. Additional information and models may also be used, such as the National Football League Scouting Combine model, which incorporates strength and cardio fitness into an evaluation score. Alternatively or additionally, cognitive ability may be incorporated into existing physical measurements and evaluation used in such models.

Based on the evaluation, there may be a determination as to whether performance modification is desired (Decision Block 22). For instance, if a young individual wishes to improve its working memory (“Yes” branch of decision block 22), then one or more options may be available. For example, the demonstrated high activity FTCD rs914246 C allele appears to encode an enzyme that is more efficient at catalyzing the synthesis of both the amino acid glutamate and forms of the vitamin folate, thereby presumably increasing levels of both substances. Thus, it may be possible to, at least temporally, modulate a level of at least one of glutamate and folate through nutritional supplementation or diet to artificially, and in effect, modulate cognitive performance.

Accordingly, and as provided in Block 24, an option may include modulating folate intake based on an individual's genotype. For example, providing a genetically-tailored diet and nutritional supplement such that folate levels are increased (increased red meat in diet or increased folate vitamin supplementation) for individuals lacking the C allele at the rs914246 DNA location or for those having the C allele but low activity levels of FTCD. Another option may include brain training procedures for those individuals lacking the C allele (Block 26). In specific embodiments, a level or extent of brain training necessary for overcoming lower glutamate and folate levels due to a lack of the C allele may be provided.

Yet other embodiments may selectively include a recommended modification of DNA to improve working memory performance (Block 28). Suitable methods for modulating DNA expression may include, for example, clustered regularly interspaced short palindromic repeats (CRISPRs). This technology had been used to functionally modify genes in human cell lines. Genetically engineering the FTCD gene, with techniques such as CRISPR/Cas9 (CRISPR associated protein 9) genome editing, to improve working memory performance and folate status is a potential application of this discovered relationship (e.g., inserting the C allele at the rs914246 DNA location for individuals needing working memory or folate treatments).

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Participants

Data from 642 cognitive aging participants was acquired and divided into two cohorts (a younger cohort with ages ranging from 18 years to 27 years and an older cohort with ages ranging from 50 years to 90 years). Each cohort was further divided into female and male cohorts. All participants provided informed consent and were screened by questionnaire for neurological and psychiatric disease. To screen for dementia, all participants of the older cohort were screened with the Mini-Mental State Exam (MMSE). Following the Alzheimer's Disease Neuroimaging Initiative (ADNI) protocol, an MMSE cut-off score of 24 was applied. All participants were assessed with a standardized neuropsychological test of episodic memory, Wechsler Memory Scale-III (WMS), logical memory, immediate recall, and delayed recall. Demographic information is reported in Table 1.

TABLE 1

| | | | WMS | WMS |
|---|---|---|---|---|
| Group | Average Age | MMSE | (Intermediate) | (Delayed) |
| Younger Female | 19.6 | N/A | 13.0 | 11.76 |
| Younger Male | 19.6 | N/A | 12.7 | 11.42 |
| Older Female | 64.8 | 28.7 | 11.6 | 9.48 |
| Older Male | 66.9 | 28.5 | 12.3 | 9.96 |

### Tasks

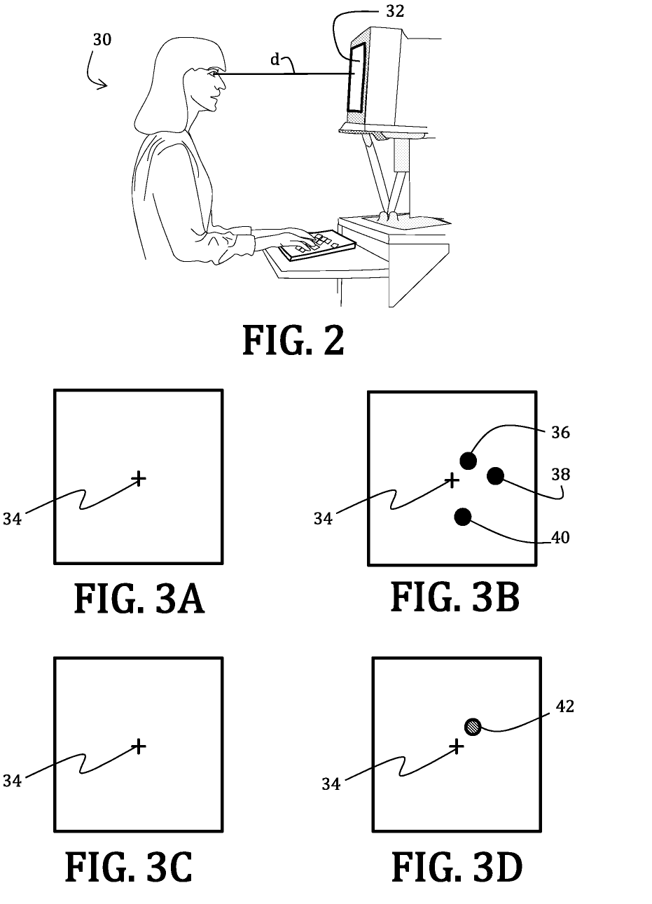

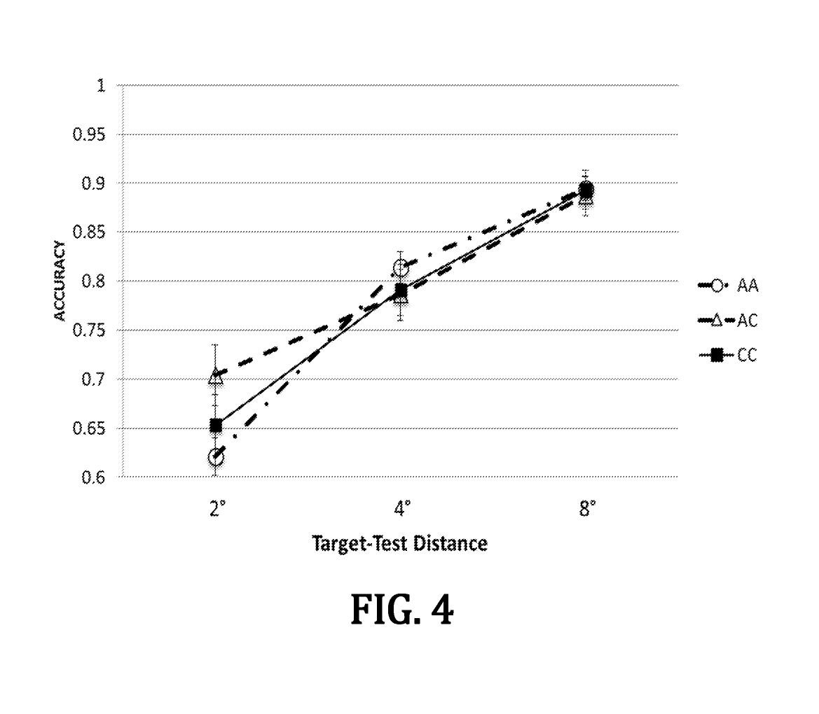

FIGS. 3A-3D illustrate a working memory task used in evaluating cognitive function of participants in all cohorts. The task included a 25 minute delayed, match-to-sample working memory task to manipulate both memory load and discrimination difficulty (distance between target and probe stimuli). More specifically, and as depicted in FIG. 2, a participant 30 was seated with their eyes located at a distance, d, from a computer screen 32 and were then given task instructions (generally, d may be 60 cm; however, other distances may also be evaluated). Every trial started with the display of a fixation cross 34 (FIG. 3A) in the center of the computer screen 32 for 1 sec. Then, one, two, or three black target dots 36, 38, 40 (0.67° visual angle in size) were then presented at random locations on the screen for 500 ms (FIG. 3B). Next, a working memory maintenance interval begins with the fixation cross 34 reappearing in the center of the display for 3 sec (FIG. 3C). Finally, in FIG. 3D, a single red probe dot 42 (0.67° visual angle in size) is displayed alone in a position that coincides with the location of one of the target dots 36, 38, 40 (match trial) or at a different location (non-match trial).

On match trials, there was a distance of 0° of visual angle between target and probe dots 36, 38, 40, 42. On non-match trials, the target and probe dots 36, 38, 40, 42 appeared randomly at differing locations with a distance of 2°, 4°, or 8° of visual angle between target and probe dot locations.

Using this setup, there are three levels of memory load (number of dot locations held in working memory, termed load 1, load 2, load 3, respectively) at each distance. At the onset of the red probe dot 42, the participant 30 has 2 sec to make a rapid decision of whether the probe dot 42 is in a location previously occupied by one of the target dots 36, 38, 40. If the location of the probe dot 42 coincides with the location of a target dot 36, 38, 40, then the participant 30 indicates this by pressing a “same” button; if the locations do not coincide, then the participant 30 indicates this by pressing a “different” button.

This task assessed a participant's ability to maintain up to three items in working memory, both when discrimination difficulty was not varied (match) and when discrimination difficulty was varied (non-match). There were a total of thirty match trials (distance 0°) and 54 non-match trials (18 trials at each distance).

### Genotyping

Single Nucleotide Polymorphisms (SNP) in the promoter region of a gene, thought to influence allele-specific transcription factor binding and gene expression, were identified: (1) rs914246 A/C SNP located 308 bp upstream of the transcription starting site (TSS) and (2) rs914245 G/T located 255 bp upstream of the TSS. Both SNPs have minor allele frequency greater than 5% and are in the same haploblock. Other SNPs within the promoter region were excluded either because of distance (too distant from the TSS, where distance exceeded 1500 bp) or minor allele frequency (less than 5%).

PROMO, a virtual transcription factor binding site in the DNA sequence identification database, predicted allele-specific transcription factor binding for rs914246 (P53, SP3, BTEB4, MYBAS1, TFIIB, EBF and ETF for the C allele; Alfm1, USF-1, NF-1 for A allele) and rs914245 (HELIOS, Olf-1 for G allele; C/EBP and Pou2F2C for T allele). The binding affinity of the listed transcription factors may be influenced by the specific allele and may change FTCD gene expression levels in certain circumstances.

Genomic material from each participant of both cohorts was obtained via buccal cell brush, and DNA was extracted using the BUCCALAMP DNA Extraction Kit from Epicentre Biotechnologies (Madison, Wis.) according to the manufacturer's instructions. Participants were genotyped in triplicate for rs914245 and rs914246 SNPs of the FTCD gene with TAQMAN Polymerase Chain Reaction (PCR) on a Bio-Rad CFX96 thermal cycler (Hercules, Calif.), which permits computerized fluorescence quenching examination of the PCR products. TAQMAN Genotyping Assay and TAQMAN GTXPRESS Master Mix were obtained from Life Technologies (Carlsbad, Calif.) and used at recommended concentrations for a 10 μL PCR reaction.

The TAQMAN Genotyping Assay was composed of two allele-specific TAQMAN MGB probes containing distinct fluorescent dyes and a PCR primer that covered the area of interest for each respective assay. The assays were validated by performing pyrosequencing of 10% of sample size on a Qiagen PyroMark Q24 machine (Venlo, NL) according to manufacturer suggested reagents and protocol. The results were analyzed with Qiagen PyroMark Q24 version 2.0.6 software (Venlo, NL), and sequenced genotypes were 100% matched with PCR results. The distribution of participants across genotypes was not significantly different from distributions previously reported for the FTCD SNPs. That is, tests for Hardy-Weinberg equilibrium revealed no significant difference: X²(2)=0.2, p>0.05 (rs914246) and X²(2)=5.5, p>0.05 (rs914245).

### Reporter Gene Assay for FTCD

A reporter gene assay (Firefly luciferase) was used to investigate the transcriptional regulation of a gene promoter coupled to expression of a reporter gene. An increase in the transcription of Firefly luciferase is easily tracked in a biological system. More specifically, treating samples with luciferin and co-factors generates luminescence for investigating the function of a targeted gene promoter. If an amount of luminescence from the experimental sample is greater than a luminescence from a control sample, then an increase in transcription or translation has occurred. Here, the FTCD promoter with the four alternative constructs of rs914245 (G/T) and rs914246 (A/C) were evaluated with the luciferase reporter gene assay.

### Plasmid Construct

The plasmid used for the reporter gene assay was constructed by Gateway Cloning technique (Life Technology, USA) for site and orientation specific recombination. Human saliva DNA samples were scrambled and the 1.6 kb FTCD promoter region that covered rs914245, rs914246, and the TSS were amplified with ATTB site attached primers by PCR and purified by gel electrophoresis. After purification, this 1.6 kb fragment was first ligated to pDNOR221 vector and then ligated to ATTB Receiver site attached PGL4.10 vector that can express Firefly luciferase activity. The sequence and orientation of the four constructs (GC, GA, TC, and TA; rs914245, and rs914246, respectively) were verified with pyrosequencing and restriction enzyme digestion.

### Dual-Glo Luciferase Assay

HEK293 cells (ATCC CRL-1573) were cultured with Minimum Essential Medium (MEM) with 10% Fetal bovine serum (FBS) at 37° C. and 5% CO₂. Approximately 1×10⁵ HEK293 cells were plated per well in a 24 well plate and incubated for 24 hrs. After incubation, the cells were transfected with 750 ng of a specified plasmid construct (GC, GA, TC, or TA constructs) and 0.25 ng of Renilla PGL4.75 control vector in each well by lipofectamine LTX with PLUS kit (Life Technology, USA) and incubated for 24 hr at 37° C. and 5% CO₂. After 24 hrs of transduction, the growth media was replaced with serum free MEM and incubated for another 24 hrs for the cells to differentiate.

Luciferase and Renilla activity were measured by a Dual-Glo Luciferase assay system (Promega, USA) and a Synergy four microplate reader (Biotek, USA). The firefly luciferase activity was normalized by Renilla luciferase, according to manufacturer guidelines.

### Analysis

From a larger sample, participants having categorized themselves as “white” according to NIH categories were evaluated for the two SNPs and working memory performance. Because of racial homogeneity of the sample, no adjustments for stratification were conducted. Participants ranged in age from 18 years to 27 years (younger cohort), and from 50 years to 90 years (older cohort).

To assess the effect of the two SNPs on working memory performance, separate mixed model analysis of variance analyses (ANOVAS) were conducted on each SNP. Match performance (target and probe dots 36, 38, 40, 42 at same location) was at a ceiling for many participants, so analyses were confined to the more difficult, non-match conditions when target and probe dots 36, 38, 40, 42 were at different locations. Between-participant factors were stated for each SNP. The within-participant factors were load and target-probe distance. Repeated measure ANOVAs were adjusted for sphericity using Greenhouse-Geisser corrections.

A given participant was included in the analyses if the accuracy for the participant was greater than 25% on all memory load and discrimination difficulty conditions. That cutoff excluded 48 participants from the original sample due to low performance by that participant under the most difficult discrimination (i.e., when the target and probe were separated by 2° of visual angle). Most of the excluded participants were in the older cohort group. Of the remaining participants, none had accuracies in the other conditions that were below 38%.

### Single SNP Analyses

As to SNP rs914245, the between-participant factors were gender (M, F), genotype (rs914245 GG, GT, TT), and age (Younger, Older). Males were more accurate than females, overall (main effect of gender, F(1, 680)=6.93, p=0.009), and the younger cohort was more accurate than the older cohort (main effect of age group, F(1, 680)=4.53, p=0.034); however, those effects did not interact (data not shown).

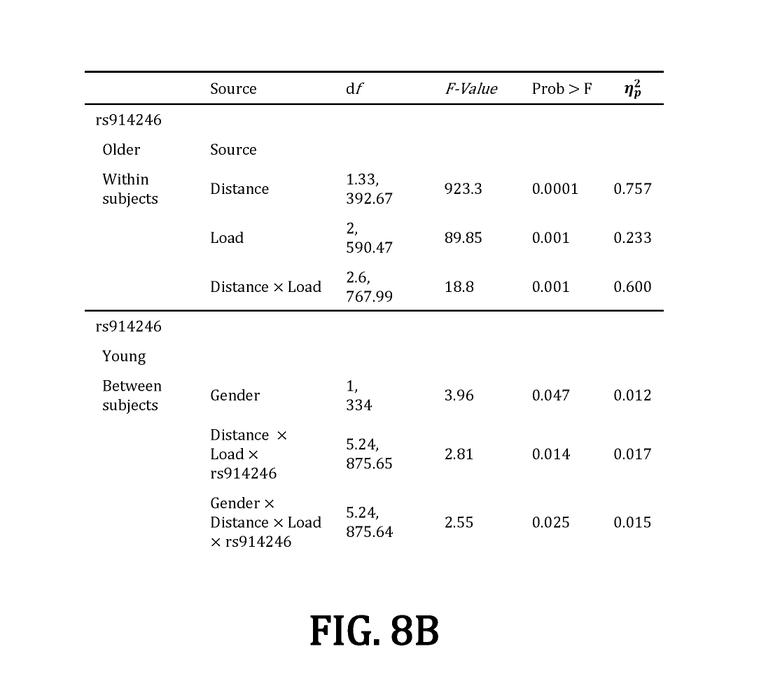

The main effect of the rs914245 genotype was not significant. There were no significant interactions involving the rs914245 genotype, although a three-way interaction correlation of distance-to-load-to-rs914245 was marginally significant (F(5.36, 1822.57)=1.972, p=0.075). Regarding the two, within-participant factors, accuracy was lowest at the shortest target-probe distance (main effect of distance, F(1.373, 933.521)=1393.112, p<0.001), and under high load (main effect of load F(1.964, 1335.810)=237.67, p<0.001). There was a significant interaction between distance and load (load-to-distance, F(2.68, 1822.565)=43.601, p<0.001). ANOVA results are provided in FIG. 8A. Based on the significant main effect of age group and the five-way interaction involving rs914246, young and older cohorts were analyzed separately in FIG. 8B.

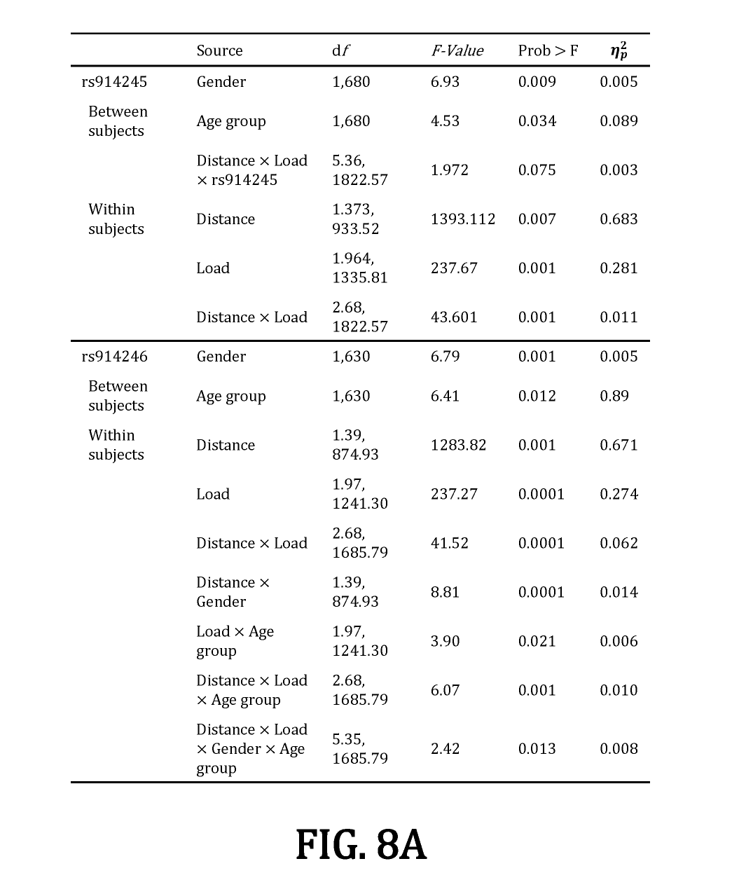

As to SNP rs914246 (FIGS. 7A and 7B), the between-subjects factors were gender (M, F), genotype (rs914246, AA, AC, CC), and age (Younger, Older). Males were more accurate than females overall (main effect of gender, F(1, 630)=6.79, p=0.001), and the younger cohort was generally more accurate than the older cohort (main effect of age, F(1, 630)=6.41, p=0.012). There was not a statistically significant interaction between age and gender.

The main effect of the rs914246 genotype was not significant. Regarding within-subject effects, there were main effects of distance F(1.39, 874.93)=1283.82, p=0.001; load F(1.97, 1241.30)=237.27, p<0.0001; and interactions of Distance-to-Gender F(1.39, 874.93=8.81, p<0.0001, Load-to-Age F(1.97, 1241.30)=3.90, p<0.021, Distance-to-Load F(2.68, 1685.79)=41.52, p<0.0001, and Distance-to-Load-to-Age Group F(2.68, 1685.79)=6.07, p<0.001. There was a significant, five-way interaction (Load-to-Distance-to-Age Group-to-Gender-to-rs914246 F(5.35, 1685.79)=2.42, p=0.013).

Based on the significant main effect of age group and the significant five-way interaction for the rs914246 SNP, younger and older cohorts were analyzed separately (FIG. 8B). For the older cohort, there were no significant effects of genotype or gender and no significant interactions involving rs914246 genotype. There were significant main effects of distance F(1.33, 392.67)=923.30, p<0.0001, load F(2.00, 590.47)=89.85, p<0.001, and a Distance-to-Load interaction F(2.60, 767.99)=18.80, p<0.001.

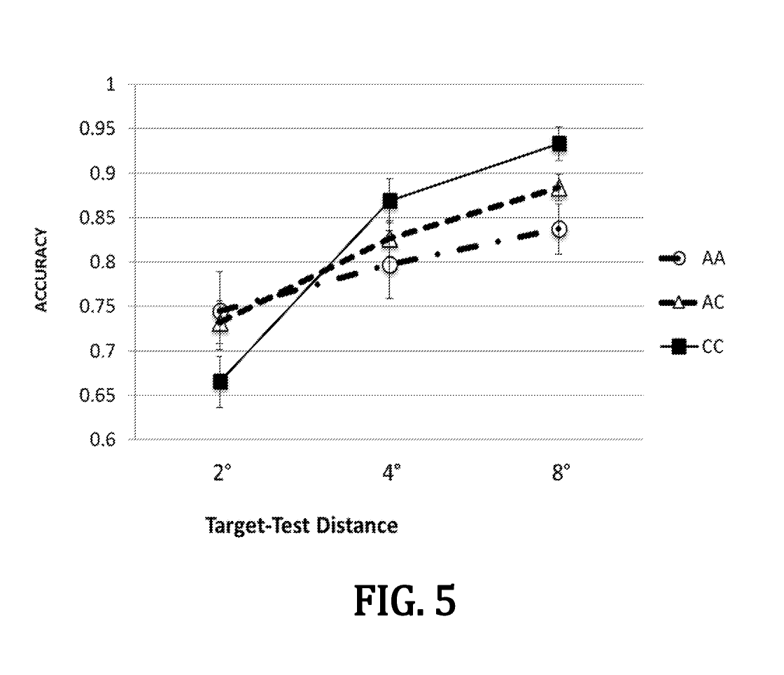

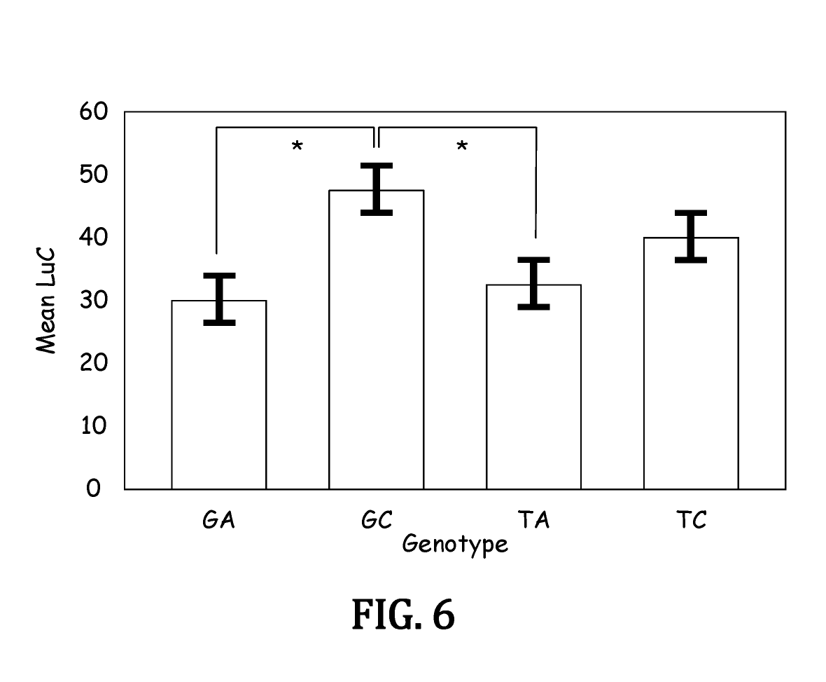

For the younger cohort, there was a significant main effect of gender F(1, 334)=3.957, p=0.047, but not of genotype. Regarding interactions involving the rs914246 genotype, there was a significant, three-way interaction of Distance-to-Load-to-rs914246 genotype F(5.24, 875.65)=2.81, p=0.014, and a significant, four-way interaction of Gender-to-Distance-to-Load-to-rs914246 genotype F(5.24, 875.65)=2.55, p=0.025. FIGS. 4 and 5 graphically illustrate the correlations between distance and rs914246 genotype at the highest load for females (FIG. 4) and males (FIG. 5), separately.

To summarize single SNP results, effects on performance of rs914246 were stronger than effects of rs914245. For the rs914246 SNP, there were both three-way and four-way interactions involving distance and load. In order to determine which of the two SNPs contributed significantly to the function of the FTCD promoter, reporter gene assay results were analyzed.

### Reporter Gene Assay

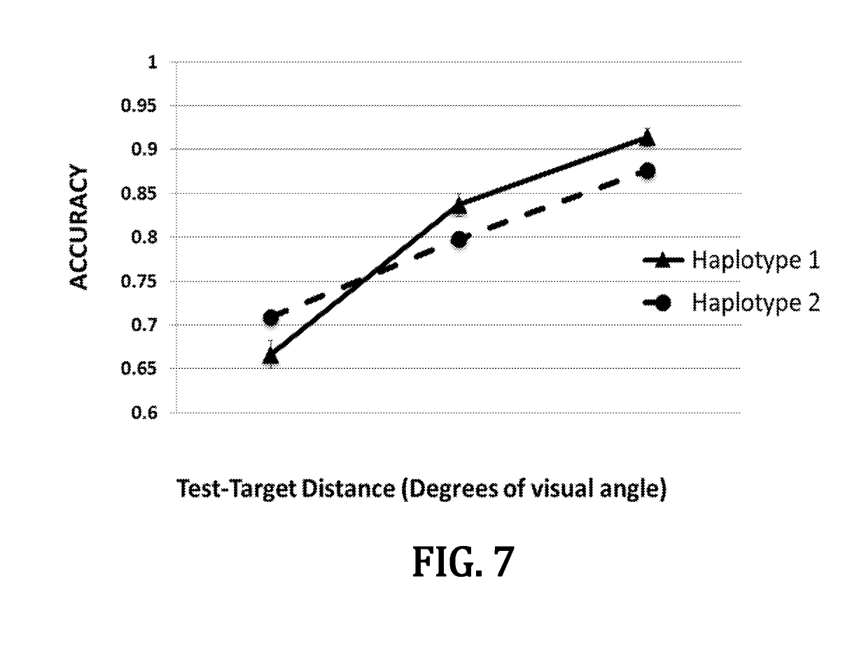

The Firefly luciferase and Renilla luciferase activities were analyzed in triplicate, and the Firefly luciferase activity was normalized to Renilla luciferase activity. The normalized mean and standard deviation of Firefly luciferase activity of GC, GA, TC, and TA constructs were 47.68±6.78, 31.02±4.36, 38.79±3.34, and 32.17±4.67, respectively, as depicted in FIG. 6. The GC and TC constructs did not differ from each other (p=0.35), and the GA and TA constructs did not differ from each other (p=0.99). The GC construct had significantly higher luciferase activity than the GA construct (p=0.02) and the TA construct (p=0.03). However, luciferase activity of the TC and the TA constructs did not differ significantly from each other (p=0.12), which would indicate that the effect of the rs914245 genotype on Firefly luciferase activity is not significant and did not contribute significantly to the function of the FTCD promoter.

Comparing the combined GC+TC constructs with the combined GA+TA constructs revealed significant differences in the Firefly luciferase activity. The C allele produced significantly higher expression of Firefly luciferase compared to the A allele (FIG. 6, F(1)=12.837, p=0.005), which indicates that the rs914246 genotype significantly regulated the Firefly luciferase activity and is a main factor regulating FTCD gene expression when participants were grouped according to combined haplotypes (1=GC or TC, 2=GA or TA).

### Haplotype Analysis

To test the effect of haplotype on working memory accuracy, a mixed model was repeatedly conducted and measured using ANOVA with haplotype, age group, and gender as between-subject factors and with distance and load as within-subject factors.

There were main effects of gender F(1, 638)=7.85, p=0.005 and age group F(1, 638)=10.25, p=0.001, but with no interaction between the two factors. The main effect of haplotype was not significant F(1, 638)=2.55, p=0.11. Accuracy was lower at shorter target-probe distances (main effect of distance, F(1.39, 881.12)=1589.50, p<0.0001) and under higher load (main effect of load F(1.97, 1248.84)=275.63, p=0.0001). There were significant interactions of Distance-to-Gender (F), Distance-to-Age (F), Load-to-Age (F), Load-to-Distance, and Age Group-to-Distance-to-Load. Haplotype and gender modulated the effect of distance (Gender-to-Haplotype-to-Distance, F(1.39, 881.13)=4.61, p=0.021) but the four-way interaction of Age Group-to-Haplotype-to-Load-to-Distance was only marginally significant p=0.05.

Based on the significant main effect of age group and the three-way interaction involving age and haplotype, follow-up ANOVAs were conducted for each age cohort separately with gender and haplotype as the between-subject factor. For the older cohort, there were no significant main effects or interactions involving haplotype or gender. There were significant main effects of load F(1.99, 691.82)=123.20, p<0.0001 and distance F(1.31, 454.19)=1301.87, p<0.0001 and a significant interaction of Load-to-Distance F(2.61, 906.53)=26.31, p<0.0001.

For the younger cohort, the males were more accurate than the females, shown in a main effect of gender F(1, 336)=7.56, p=0.006. Gender interacted with haplotype at a marginal level of significance (p=0.091). There were significant main effects of load F(1.93, 646.17)=184.02, p<0.0001 and distance F(1.46, 490.16)=505.39, p<0.0001. Distance interacted with gender (Distance-to-Gender, F(1.46, 490.16)=4.58, p<0.02) and with haplotype (Distance-to-Haplotype, F(1.46, 646.17)=4.46, p=0.022). FIG. 7 graphically illustrates the significant, three-way interaction of Distance-to-Haplotype-to-Load F(2.62, 880.82)=5.03, p<0.001. This interaction was modulated at a marginal level by gender (Gender-to-Haplotype-to-Distance-to-Load, F(2.62, 880.82)=2.65, p=0.055).

To analyze the significant, three-way interaction of Distance-to-Haplotype-to-Load, a main effects analysis was conducted by comparing haplotype at each combination of load and distance. The analysis showed the haplotype groups differing significantly at each level of distance, but only under the highest load (three locations). FIG. 6 shows that haplotype 1 (containing the C allele, which produced significantly higher Firefly luciferase expression) was associated with higher accuracy under the two easy discrimination conditions, but with lower accuracy under the hardest discrimination conditions. Results of the effects analysis were as follows: distance 1, F(1,336)=4.23 p=0.040; distance 2, F(1,336)=4.56, p=0.033; and distance 3, F(1,336)=8.14 p=0.005.

Because of the main effect of age group and the three-way interaction involving age and haplotype, the younger and older cohorts were analyzed separately.

The effect of haplotype on episodic memory (the memory of autobiographical events) was analyzed using the Wechsler Memory Scale, logical memory subtest. A mixed-model ANOVA was conducted on immediate and delayed responding (within subjects), with gender (M, F) and age group (younger, older), and haplotype (1,2) as between subjects factors. Overall, older cohort participants were less accurate than younger cohort participants (main effect of Age Group, F(1, 692)=17.67 p=0.0001). There was not a main effect of gender or haplotype. As expected, accuracy was higher for immediate recall than delayed recall, producing a main effect of WMS, F(1,1787.11)=377.73, p=0.0001. The immediate/delayed factor did not interact with age group or haplotype. Other tested effects did not reach statistical significance.

The rs914246 C allele exerted differential effects as a function of discrimination difficulty, but only under high working memory load. The working memory task varied the distance between target and probe. Under the closest distance (about 2° of visual angle), accuracy was sharply lower as compared to the two longer distances. The C allele carriers performed more accurately than the non-carriers under the two easier discrimination conditions, but less accurately under the hardest discrimination condition. Due to the bifunctional nature of the FTCD gene, it could not be determined whether modulation of accuracy in C allele carriers is due to the production of glutamate, folate, or both. Non-linear effects on working memory performance have also been observed in the dopamine system.

During working memory performance, dopamine D1 receptors in PFC showed a dose-response function that has been characterized as an “inverted-U,” such that both low and high levels of stimulation impair task performance. The present findings show a different, non-linear pattern on working memory performance but only under high memory load in the younger cohort. Previously it was determined that COMT and DBH genes altered working memory performance only in older individuals and only under high discrimination demands, regardless of working memory load. Altogether, this evidence suggests that the ability to retain and manipulate information in memory may depend on several distinct mechanisms.

As working memory has been strongly associated with dopamine neurotransmission, the complex effects observed could be due, in part, to interactions between dopamine and glutamate neurotransmission systems. Animal evidence of synergistic interactions between glutamate and dopamine systems has been shown and, specifically, combined DI dopamine and glutamate receptor blockade on dendritic spines of GABA neurons selectively affected executive function. It has also been shown that combined dopamine and glutamate blockade selectively impaired executive function while separate administrations had no effect. In humans, interactions between genes that influence dopamine and glutamate transmission (dopamine transporter [DAT] and the D-Amino acid oxidase activator [DAOA], with effects on dopamine and glutamate transmission, respectively) have been found to modulate fMRI brain activation patterns in a number of regions.

While males performed better overall than females on the spatial working memory task, that effect did not interact consistently with haplotype. A gender difference is consistent with a large body of evidence showing that males perform better than females on spatial working memory. A recent meta-analysis of 98 samples across lifespan found the advantage emerges in adolescence and persists into old age. Moreover, there is neurocognitive evidence of gender differences in patterns of network activation mechanisms during a spatial working memory task. Adolescent girls showed de-activation of the default-mode network (linked to retrieval from long-term memory) while boys showed activation of frontal cortex regions, which are linked to working memory. The gender-differences in performance and activation patterns were not found to be related to endogenous testosterone levels.

In summary, variation in FTCD affected spatial working memory in younger cohorts but not older cohorts, suggesting some age-related change in the signaling pathways involved. These findings extend previous work with genes in the dopamine pathways to a gene outside the dopamine pathway, but these results demonstrate an age-related decrease rather than the conventional increase. These findings that genetic variants affect memory performance have real-world implications. Evidence of detectable effects of normal genetic variation in the dopamine pathway on memory performance in a range of tasks, and a real-world executive function task, suggests that there is a potential not only to select individuals for a given type of task, but also to tailor task training for individuals based on genotype.

As described herein, the rs914246 variant of the FTCD gene modulated accuracy in a spatial working memory task. A reporter gene assay revealed the C allele of the rs914246 variant to be the main factor regulating FTCD gene expression. The C allele of the rs914246 variant in the FTCD gene was also found modulated working memory performance.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A method of predicting cognitive performance for a person, the method comprising:
acquiring a genetic sample from the person;
determining an SNP of the rs914246 of the FTCD gene of the person;
comparing the determined SNP against a cognitive model; and
based on the comparison, returning a predicted cognitive performance.

2. The method of claim 1, wherein the determination is of a C allele SNP.

3. The method of claim 1, wherein the determination is of an A allele SNP.

4. The method of claim 1, further comprising:
recommending a diet of at least one of increased folate and increased glutamate for the person.

5. The method of claim 1, wherein the comparison includes correlation to physical measurements of the person.

6. The method of claim 1, further comprising:
modulating expression of a C allele SNP of the rs914246 of the person.

7. The method of claim 1, further comprising:
recommending cognitive training for the person.

8. The method of claim 1, further comprising:
recommending DNA modification for the person.

9. A method of predicting cognitive performance for a person, the method comprising:
acquiring a genetic sample from the person,
determining a C allele SNP of rs914246 of the FTCD gene of the person,
comparing the determined SNP against a cognitive model, and
based on the comparison, returning a predicted cognitive performance.

10. The method of claim 9, further comprising recommending a diet of at least one of increased folate and increased glutamate for the person.

11. The method of claim 9, wherein the comparison includes correlation to physical measurements of the person.

12. The method of claim 9, further comprising:
modulating expression of the C allele SNP of the rs914246 of the person.

13. The method of claim 9, further comprising:
recommending cognitive training for the person.

14. The method of claim 9, further comprising:
recommending DNA modification for the person.

15. A method of predicting cognitive performance for a person, the method comprising:
acquiring a genetic sample from the person,
determining an A allele SNP of rs914246 of the FTCD gene of the person,
comparing the determined SNP against a cognitive model, and
based on the comparison, returning a predicted cognitive performance.

16. The method of claim 15, further comprising:
recommending a diet of at least one of increased folate and increased glutamate for the person.

17. The method of claim 15, wherein the comparison includes correlation to physical measurements of the person.

18. The method of claim 15, further comprising:
modulating expression of the C allele SNP of the rs914246 of the person.

19. The method of claim 15, further comprising:
recommending cognitive training for the person.

20. The method of claim 15, further comprising:
recommending DNA modification for the person.
